(19) United States
(12) Patent Application Publication
(10) Pub. No.: US 2008/0274125 A1
(43) Pub. Date: Nov. 6, 2008

(54) Human Stem Cell Lines Derived From Es Cells and Uses for Production of Vaccines and Recombinant Proteins

(75) Inventor: Fabienne Guehenneux, Le Temple de Bietagne (FR)

Correspondence Address: BUCHANAN, INGERSOLL & ROONEY PC, POST OFFICE BOX 1404, ALEXANDRIA, VA, 22313-1404, US

(73) Assignee: VIVALIS, ROUSSAY (FR)

(21) Appl. No.: 11/792,510

(22) PCT Filed: Dec. 8, 2005
(86) PCT No.: PCT/EP2005/056608

§ 371 Date: Apr. 14, 2008

(30) Foreign Application Priority Data
Dec. 8, 2004 (FR) 0413058

(51) Int. Cl. A61K 39/00; C12N 5/06; C12N 7/00; A61P 37/00; C12Q 1/02; C12P 21/04

(52) U.S. Cl. 424/184.1; 435/385; 435/366; 435/235.1; 435/69.1; 435/29

(57) ABSTRACT

The present invention concerns the field of biology and virology. In particular, the invention concerns a method for obtaining human cell lines, in particular human stem cells derived from human embryonic stem cells, the method comprising separation from the serum, the feeder layer and at least one growth factor. The cell lines are capable of proliferating indefinitely in a basic culture medium. The invention also concerns the use of the cells derived from such cell lines for virus replication, and more particularly for producing human or veterinary vaccines, as well as for producing recombinant proteins of therapeutic interest.

The present invention relates to the field of biology and virology. In particular, the invention relates to a process for obtaining human cell lines, especially human stem cells derived from human embryonic cells, comprising separation of the serum, the feeder layer and at least one growth factor. These lines are capable of proliferating indefinitely in a basic culture medium. The invention also refers to the utilisation of cells deriving from such lines for virus replication, and more particularly for producing human or veterinary vaccines, as well as for producing recombinant proteins of theracanic interest.

Historically, the vaccine industry has used, and still uses today, embryonic eggs to produce a number of vaccines such as the vaccine against the human flu. However, this production system based on embryonic eggs has numerous limitations such as: (i) a variable biological quality of the eggs, due to the presence of accidental agents (virus, toxins, . . . ) or sterility problems, (ii) the absence of constancy in providing eggs throughout the year, (iii) the absence of flexibility in producing eggs in the event of a sudden increase in demand (i.e. in the case of a sudden epidemic or pandemic), (iv) significant financial cost. A solution made by the pharmaceutical industry to the production problems in eggs consisted of producing vaccines on cellular culture. In fact, the virus and viral vectors can be replicated and cultivated in a large number of primary diploid cells, such as monkey kidney cells, cattle kidney cells, hamster kidney cells and the chicken embryo fibroblasts. For example, replication and propagation of certain viruses such as poxvirus are carried out in cultures of primary cells of chicken embryo fibroblasts (CEF for “chicken embryo fibroblasts”). However these primary cells suffer from numerous disadvantages, such as contamination by accidental agents and/or pathogens (bacteria, mycoplasms, yeasts, . . . ), variable quality of cells in culture, different sensibilities to variants of the same virus, low titres viral, high virus production costs, the necessity to re-establish primary cells at each vaccine preparation, the necessity to utilise sera of animal origin to complement the culture medium, with the inherent risks of contamination by mycoplasms, viruses or agents of the bovine spongiforme encephalitis (BSE) and finally difficulties in obtaining and preparing such cells in culture.

This is the reason for which the use of continuous immortalised cellular lines has been put forward for virus or viral vector replication. Accordingly, continuous lines of animal origin such as for example the MDCK cellular line derived from the Madin-Darby dog kidney (Tobita et al., 1975, Med. Microbiol. Immunol. 162:9-14), the VERO cellular line derived from the African green monkey kidney (U.S. Pat. No. 5,824,536), the BHK21 cellular line derived from baby hamster kidney were established. Human lines such as PER.C6 (WO 01/38362) were also developed for vaccine production. However, the continuous cellular lines currently available do not give total satisfaction. So, due to their specificity of kind certain cellular lines do not replicate certain animal viruses, or do so poorly. Also, certain cellular lines do not achieve economically profitable viral productivity. Moreover, industrial development of certain lines is at times difficult, since it is necessary to gave available cells capable of growing in aseric medium and in suspension. Also, certain of these continuous lines are likely to not satisfy regulatory requirements, as they have an unstable and abnormal caryotype, and/or are transformed genetically and/or are tumorigenic “in vivo”. These regulatory considerations constitute a particularly important point when it is planned to use a cellular line from which vaccines will be isolated. Finally, the existence of strong industrial protection on the most efficient continuous cellular lines should also be mentioned. For these various reasons, pharmaceutical and veterinary enterprises in the vaccine field are researching novel continuous cellular lines not exhibiting these disadvantages.

The problems encountered with continuous cellular lines in the vaccine field are also encountered in the field of the production de recombinant proteins in continuous cellular lines. In addition to the regulatory aspects associated with the security and stability of continuous lines, the major limitation encountered is the productivity of the cell. It is in fact necessary to have a cell capable of growing in suspension indefinitely in a medium without serum. Overall, analysis of the prior art reveals an unfulfilled and long-awaited need to develop a cell capable of assuring replication of virus and viral vectors, but also production of recombinant proteins, in a cellular system not exhibiting the disadvantages of existing production systems such as embryonic chicken eggs, primary diploid cells or continuous cellular lines currently available.

This is the problem put forward for solving the present invention by proposing a process for obtaining lines of human stem cells adapted to utilisation by the pharmaceutical industry. In fact, different documents of the prior art describe the cultivation and keeping in culture of primary human embryonic stem cells. Thomson et al. (1995, Proc. Natl. Acad. Sci USA 92:7844; U.S. Pat. No. 5,843,780) were the first to successfully cultivate stem cells of primates. They are subsequently derived from lines of human embryonic stem cells from blastocysts (1998, Science, 282:114). Gearhart et al. derived cellular lines of germinal embryonic human cells (hEG) from gonadic foetal tissue (WO 98/43679). However, to this day the primary human embryonic cells stem described are not adapted to their industrial utilisation, given the necessity of cultivating them in adherence in complete medium in the presence of growth factors, animal serum and feeder cells. The present invention seeks to solve this problem by providing a process for producing lines of human stem cells, said process comprising the following stages:

a) culture of human stem cells in a complete culture medium containing all the factors enabling their growth, a feeder cell layer, preferably inactivated by irradiation, and complemented by serum;

b) successive passes by modifying the culture medium to produce progressive or total separation into feeder cells, and/or growth factors, and/or serum. The separation sequence of the culture medium is preferably: separation into growth factors, then separation into feeder cells, then separation into serum;

c) setting up adherent and non-adherent human cellular lines capable of proliferating in a basic culture medium in the absence of feeder cells, and optionally in the absence of exogenic growth factors and/or containing low seric concentration, or even not containing serum in the culture medium.

The process according to the invention further comprises the preliminary stage of obtaining a population of human stem cells. In terms of the present invention stem cell is understood to mean an undifferentiated cell, issuing from the embryo, the foetus or the adult. The stem cell is characterised by its capacity of auto-renewal (that is, identical multiplication to produce new stem cells), of differentiation in certain culture conditions (so as to engender specialised cells) and of proliferation in culture. According to a first embodiment, the human stem cell according to the invention is a totipotent stem cell originating from the first divisions of the fertile egg to the fourth day of development. These totipotent stem cells potentially have the capacity to regenerate a complete individual. According to a second embodiment of the invention, the human stem cell according to the invention is a stem pluripotent cell, also known as ES embryonic stem cell. The ES cells number as many as one hundred cells in the internal mass of the embryo at the blastocyst stage (from the 5th to the 7th day after fertilisation). The ES cells are capable of participating in the formation of all the tissues of the organism (more than 200 cellular types). According to a third embodiment, the human stem cell according to the invention is a multipotent stem cell. These cells present in foetal or adult tissue have the capacity for auto-renewal and can give birth to several types of cells. These cells are engaged in a specific tissue program, such as haematopoietic stem cells of bone marrow and cord blood. According to a fourth embodiment, the human stem cell according to the invention is a unipotent stem cell. These cells generate only a single type of differentiated cells by retaining a certain capacity for auto-renewal and proliferation (examples: hepatocytes of the liver, keratinocytes of the skin, . . . ). According to a fifth embodiment, the human stem cell according to the invention is an intermediary progenitor cell. These cells have no or little capacity for renewal and are divided solely into differentiated cells. According to a preferred embodiment of the invention, the stem cell according to the invention is a human embryonic stem cell (ES) isolated or propagated from a human blastocyst.

By way of preference, the human stem cell according to the invention was not transformed chemically or by means of a biological agent (virus, nucleic acid, . . . ).

According to a preferred embodiment, the present invention relates to a process for providing continuous lines of non-transformed, undifferentiated human stem cells capable of proliferating indefinitely in culture, characterised in that said process comprises the following stages:

a) culture on a layer of feeder cells of primary human stem cells in a complete culture medium comprising at least:
animal serum;
an exogenic growth factor selected from FGF (Fibroblast Growth Factor), stem cell factor (SCF) and the IGF1 (Insulin-like growth factor 1);
an exogenic growth factor selected from the receptor ligands which can form a hetero-dimer with glycoprotein gpl30. Examples of ligands are Leukaemia Inhibitory factor (LIF), interleukine 11 (ILI 1), interleukine 6 (IL6), interleukine receptor 6 (IL6R), ciliary neurotrophic factor (CNTF), Foncostatine, cardiotrophine;

b) successive passes in a culture medium identical or different, so as to obtain separation of the cellular feeder layer, total or partial separation of said exogenic growth factors and total or partial separation of the serum;

c) optionally, selection of cellular colonies having compact morphologies and composed of cells having a heightened nucleo-cytoplasmic ratio and a preeminent nucleole;

d) setting up of said continuous lines of human stem cells derived from embryonic cells capable of growing in the absence of feeder cells.

Preferably, said lines obtained in stage d) are capable of growing in a culture medium totally devoid of growth factors. Even more preferably, said lines obtained in stage d) are capable of growing in a culture medium totally devoid of growth factors and totally devoid of serum.

Said primary human stem cells are selected from totipotent stem cells, pluripotent stem cells, multipotent stem cells, unipotent stem cells, intermediary progenitor cells. Preferably, said primary human stem cell is a pluripotent stem cell, that is, an embryonic stem cell (ES).

The process according to the invention can further comprise a sub-stage al) of stage a) consisting of dissociating the cellular clusters formed in culture, characterised in that dissociation is done enzymatically and/or mechanically. When performed enzymatically, trypsine, pronase or collagenase are preferably used. Mechanical dissociation is done with a scraper or by way of a slicing object for fractionating the compact clusters from cells into small cellular groups facilitating amplification of the cultures after transplanting or individualising the cells making up the cellular clusters.

The terms “growth factor” or “factor enabling their growth”, used variously in the present invention signify a chemical or biological substance (in general this is a peptide or a protein) necessary for survival and growth of human cells in culture. It is possible to schematically distinguish two families of growth factors: cytokines and trophic factors. Cytokines are essentially proteins whereof the action is done via the receptor which is associated with the protein GP 130. Therefore, LIF (Leukaemia Inhibitory Factor), Finterleukine 11 (Il-H), interleukine 6 (Il-6), interleukine receptor 6 (Il-6R), Ciliary Neurotrophic Factor (CNTF), oncostatine and cardiotrophine have an action mode similar to recruitment at the level of the receptor of a specific chain and the combination of the latter with protein GP 130 in monomeric or sometimes heterodimeric form. Trophic factors are principally SCF, IGF-1 and FGF.

“Complete culture medium” is understood to mean a base medium complemented with vitamins, nutrients, mineral salts, growth factors, serum, and diverse compounds for assuring optimised growth of cells in culture. According to the present invention, a “base medium” signifies a medium whereof the formulation ensures survival of the cells in culture, and minimal growth. Examples of base media (or basic media) are for example the medium BME (Basai Eagle Medium), MEM (Minimum Eagle's Medium), medium 199, DMEM (Dulbeco's Modified Eagle Medium), GMEM (Glasgow Modified Eagle's Medium), DMEM-HamF12, Ham-F12 and Ham-F10, Iscove's Modified Dulbecco's Medium, MacCoy's 5 A medium, RPMI 1640. The base medium comprises mineral salts (for example: CaCl₂, KCl, NaCl, NaHCO₃, NaH₂PO₄, MgSO₄, . . . ), amino acids, vitamins (thiamine, riboflavin, folic acid, calcium panthothenate, . . . ) and other components such as glucose. It can be necessary to complement the base medium with at least one of the following compounds: animal serum, L-glutamine, sodium pyruvate, beta mercaptoethanol, amino acids, vitamins, growth factors for generating a complete medium.

According to a preferred embodiment of the invention, said complete medium at stage a) comprises serum, and at least FGF and LIF. According to another embodiment said complete medium at stage a) comprises serum, FGF, LIF and at least one compound selected from IL-6, IL6R, IL11, CNTF and IGF1. According to yet another embodiment, said complete medium at stage a) comprises serum, FGF, LIF, IL6, IL6R, IL11, CNTF and IGF1. The FGF according to the invention is preferably selected from basic FGF, FGF3 and FGF4. The concentration of growth factors in the base medium is between around 0.01 to 10 ng/ml, preferably 0.1 to 5 ng/ml, and preferably around 1 ng/ml.

According to a preferred embodiment, the cellular feeder layer of stage a) is composed of fibroblasts selected from primary human fibroblasts, human fibroblasts set in line, primary mammal fibroblasts, mammal fibroblasts set in line. These are preferably mammal fibroblasts, more particularly mouse fibroblasts set in line, preferably STO cells transformed or not. The cellular feeder layer is preferably inactive. It can be inactivated chemically by mitomycin processing for example, or physically by exposure to physical rays such as X rays or gamma rays.

The present invention rests on the discovery that passing a medium of base cellular culture complemented by growth factors containing animal serum and feeder cells to an aseric medium deprived of growth factors can be completed only by simply removing them from the basic culture medium. The deprivation process according to the invention requires completing the deprivation of growth factors, feeder cells and serum sequentially and progressively.

To successfully carry out deprivation of growth factors, it is important that the complete starting medium in which the human stem cells taken from an individual are cultivated comprises at least two, at least 3, at least 4, at least 5, at least 6, at least 8 different growth factors so that when deprived of one of its factors the cell can adapt and compensate for this deprivation by developing an alternative metabolic path. When the medium comprises at least two different growth factors these are preferably LIF and FGF.

Modification of the culture medium at stage b) of the process of the invention, for the purpose of obtaining progressive or total removal of growth factors, serum and/or feeder cell layers can be done simultaneously, successively or separately over time. Said instances of separation into cellular feeder layer, exogenic growth factors, and serum are carried out successively or staggered over time, according to a separation sequence selected from:
feeder cells/serum/growth factors;
feeder cells/growth factors/serum;
serum/growth factors/feeder cells;
serum/feeder cells/growth factors;
growth factors/feeder cells/serum;
growth factors/feeder cells/serum.

The serum utilised is preferably animal serum, more preferably foetal calf serum. Alternatively, the serum can be a substitute of serum such as currently marketed by certain companies (ex. KO SR by GIBCO-BRL).

According to a preferred embodiment, the separation sequence is 1) separation into growth factors, 2) separation into feeder cells and 3) separation into serum.

During stage a) the human stem cells are cultured for around 3 to 40 passes in complete medium, then the complete medium is progressively and sequentially voided of growth factors (stage b). Depletion for each growth factor is preferably carried out directly in a single stage, from one pass to the other. Alternatively, depletion of growth factor is carried out gradually, by progressive decrease with each pass of the concentration of the growth factor in the complete culture medium. According to a preferred embodiment depletion of growth factors is carried out simultaneously for at least two growth factors. Alternatively, depletion of growth factors is carried out sequentially growth factor after growth factor. In a preferred manner, LIF is removed first and directly from the complete culture medium, then after several passes in culture in a complete medium deprived of LIF, the FGF is in turn removed directly from the culture medium. In a preferred manner, separation into exogenic growth factors is total. The medium is normally totally devoid of growth factors approximately by passes 20 to 40.

Usually, deprivation of feeder cells is carried out after deprivation of growth factors. Deprivation of feeder cells is progressive and carried out over several passes. The human stem cell is in general sown in flasks at a concentration lower than that carried out in stage a) at approximately 4×10⁴ cells/cm² up to 5×10⁴ cells/cm². The feeder cells are sown in a flask at approximately 4×10⁴ cells/cm². Progressively, the concentration of feeder cells in the flasks drops. In practical terms, the same concentration of feeder cells is utilised for 2 to 5 passes, then the concentration of feeder cells drops over 2 to 5 passes, and so on. By way of example, the flask is sown at approximately 4×10⁴ cells/cm², then approximately 3×10⁴ cells/cm², then approximately 2×10⁴ cells/cm², then approximately 1.5×10⁴ cells/cm², then approximately 10⁴ cells/cm², then approximately 0.5×10⁴ cells/cm². Finally the flask is sown with 6×10⁴ human cells/cm² at 10⁵ human cells/cm² in the absence of feeder cells. In the hypothesis where the human cells are not in good condition after decreases in concentration of feeder cells in the flask, the human cells are cultivated over several extra passes at the same concentration of feeder cells in the flask prior to continuing deprivation of feeder cells. At stage b), a change in the nature of the plastic material of the boxes of cellular culture used is carried out simultaneously, successively or staggered over time with the separation of the cellular feeder layer. Said plastic material used is treated specifically so as to benefit the non-ionic or hydrophobic interactions, to diminish adhesion of the cells to said material. Stage b) comprises at least 30, at least 50, at least 75, at least 100, at least 125, at least 130 successive passes in culture.

The process of obtaining lines of human stem cells according to the invention further produces lines capable of growing aseric medium. The seric separation of the cells according to the invention is done by modifying or changing the culture medium of the cells so as to obtain total separation of serum, either by progressive dilution, direct separation, or progressive separation. This method selects clones which adapt to these new but more and more drastic culture conditions, until stable cellular lines are obtained which are capable of growing medium devoid of serum or in a medium without serum.

The basic culture medium of stage c) preferably comprises a low concentration of serum (that is, a seric concentration in the culture medium of less than or equal to 5%). The process according to the invention optionally comprises the additional stage c) bis of changing the culture medium of stage c). The medium used in stage c) bis is therefore selected from:
the base medium (i) complemented by serum and diluted with a new aseric medium (ii). Next, the human cells are cultivated by successive passes in a medium (i) in which the proportion of medium without serum (ii) is progressively increased until the base medium (i) complemented by serum (progressive dilution) completely disappears;
a new medium without serum (ii) complemented by serum. Next, the human cells are cultivated by successive passes in a medium (ii) in which the proportion of serum is progressively diminished, until an aseric medium is obtained (progressive separation);
a new medium without serum (ii) not complemented by serum. Then the human cells are directly cultivated in the aseric medium (ii) (direct separation).

Said separation into serum is done by implementing a process selected from progressive dilution, progressive separation or direct separation. According to a preferred embodiment, separation into serum is carried out by progressive separation.

According to the present invention, the term “aseric medium” or “medium without serum” (SFM) means a ready-to-use cellular culture medium, that is, requiring no addition of serum for survival and cell growth. The medium is not necessarily defined chemically and can contain hydrolysats of varying origin, such as hydrolysats of vegetable origin, for example. Said medium SFM is preferably qualified “without any compound of origin animal”, that is, it contains no components of animal or human origin (FAO statute: “free of animal origin”). In the aseric medium, the native proteins of the serum are replaced by recombinant proteins. Alternatively, the SFM medium according to the invention contains no protein (medium PF: “protein-free”) and/or is defined chemically as a CDM medium: “chemically-defined medium”). The SFM medium offers a number of advantages: (i) the first is its aptitude to satisfy regulatory demands (there is no risk of contamination by biological agents such as prions or animal viruses); (ii) optimisation of the purification process; (iii) best reproducibility of performances of the cellular culture since the medium is better defined. Examples of aseric SFM media being marketed are VP SFM (InVitrogen Ref. 11681-020, catalogue 2003), Opti Pro (InVitrogen Ref. 12309-019, catalogue 2003), Episerf (InVitrogen Ref. 10732-022, catalogue 2003), Pro 293 S-CDM (Cambrex Ref. 12765Q, catalogue 2003), LC 17 (Cambrex Ref. BESP302Q), Pro CHO 5-CDM (Cambrex Ref. 12-766Q, catalogue 2003), HyQ SFM4-CHO (Hyclone Ref. SH30515-02), HyQ SFM4-CHO-Utility (Hyclone Ref. SH30516.02), HyQ PF293 (Hyclone Ref. SH30356.02), HyQ PF Vero (Hyclone Ref. SH30352.02), Ex cell 293 medium (JRH Biosciences Ref. 14570-1000M), Ex cell 325 PF CHO Protein free medium (JRH Biosciences Ref. 14335-1000M), Ex cell VPRO medium (JRH Biosciences Ref. 14560-1000M), Ex cell 302 serum free medium (JRH Biosciences Ref. 14312-1000M).

The process of obtaining human stem cells as described hereinabove can also comprise an additional stage in which the cells obtained in stage c) are subjected to selection and adaptation in a suitable culture medium to produce cellular clones useful for the production of biological substances on a large scale.

Human stem cells, preferably human stem cells derived ES human cells, established by using the process of the invention, are preferably cells which proliferate in aseric medium, in the absence of feeder cells and do not require the addition of growth factors in the culture medium. Novel lines of human stem cells, derived preferably from stem embryonic cells, obtained by the process according to the invention can be maintained in culture in vitro for a long period, that is, more than one hundred passes.

By way of advantage, stem embryonic cells obtained at stage d) are capable of proliferating for at least 50 days, at least 100 days, at least 150 days, at least 300 days in culture and preferably at least 600 days in culture. These 600 days are in no way a limit, since the cells obtained will always be living after this date. Because of this, the stem cells according to the invention are considered as being capable of growing indefinitely in culture in a basic culture medium comprising no exogenic growth factors, serum and/or inactivated feeder cell layers. The expressions “lines” or “continuous lines”, terms employed variously in this patent, mean that the cell population is capable of growing or proliferating indefinitely in culture in vitro, while essentially retaining the same morphological and phenotypic characteristics. “Undefined growth in culture” is understood to mean a property of cellular lines in culture allowing propagation over the long term. This characteristic opposes those presented by the majority of normal diploid cells isolated and cultivated in vitro, such as cells known as “primary” when enter senescence after multiple passes. According to the present invention, the term “undefined growth” comprises a culture of at least 30 days, at least 60 days, preferably at least 6 months, most preferably at least one year.

The stem cells established according to the invention are preferably small, round, well individualised with a doubling period of between 20 and 40 hours, preferably between 24 and 30 hours. The cells obtained by the process according to the invention are at least at pass p60, at least at pass p70, at least at pass p80, at least at pass p100, at least at pass p120, at least at pass p140 or more. The cells established by the process according to the invention have the aptitude of proliferating for at least 50 days, at least 100 days, at least 150 days, at least 300 days in culture and preferably at least 600 days in culture in a base medium such as DMEM, GMEM, HamF12, Optipro (GIBCO-BRL) or MacCoy supplemented with various additives currently used by the expert. Examples of additives are non-essential amino acids, vitamins, sodium pyruvate, beta-mercaptoethanol, etc.

The cells of the cellular line obtained by the process according to the invention are derived from human stem cells, preferably embryonic (ES), and have at least one of the following characteristics:
proliferate indefinitely in culture in a culture medium deprived of cellular feeder layer, optionally of serum and optionally of exogenic growth factors; and
retain a normal diploid caryotype which is not altered by prolonged cellular culture; and
exhibit a significant nucleo-cytoplasmic ratio; and
retain the capacity to differ for forming at least one differentiated cellular type selected from a cellular type of mesodermic, ectodermic and endodermic origin; and
express at least telomerase and alkaline phosphatase. The line of cells according to the invention is characterised in that the cells of said line further express the transcription factor Oct3/4 and have reactivity with at least one of the specific antibodies selected from the antibodies directed against SSEA4, TRA 1-60, TRA 1-81. The line of cells according to the invention is further characterised in that the cells of said line exhibit no reactivity with the antibody directed against SSEA1.

The line of cells according to the invention preferably has a normal caryotype selected from 46 XX and 46 XY).

The doubling time of the human stem cells obtained by the process according to the invention is characterised by a shorter doubling time than the doubling time of the primary human stem cells of stage a) according to the process of the invention. The doubling time of the stem cells obtained by the process according to the invention is approximately between 20 and 40 hours, preferably between 24 and 30 hours.

Of course, the process described here provides cellular clones derived from cells obtained from these lines. These clones are cells which are genetically identical to the cells from which they derive by division.

The cells according to the invention are capable of proliferating in adherence on the support, but they can also be adapted for culture in suspension. The cells according to the invention preferably have all the characteristics mentioned above.

The invention also aims to cover the cells according to the invention which have been modified genetically either stably or transitorily by implementing techniques well known to the expert. The genome of said cell can thus be modified by:
i. insertion of an isolated pre-selected DNA sequence; or
ii. substitution of a fragment of the cellular genome by an isolated pre-selected DNA sequence; or
iii. deletion of an isolated pre-selected DNA sequence; or
iv. inactivation of an isolated pre-selected DNA sequence.

The invention also aims to cover differentiated human cell lines obtained from the stem cells obtained by the process according to the invention. Said differentiated cell is selected preferably from neural cells, oligo-dendrocytes, glial cells, haematopoietic cells, exocrine cells, endocrine cells, epithelial cells, endothelial cells, cardiac muscle, skeletal muscle, bone marrow, fibroblasts, adipocytes, cartilage cells, bone cells.

According to a particular embodiment of the invention, the human stem cells obtained by the process of the invention are utilised as a drug in cellular therapy in vivo, especially for the treatment of neuro-degenerative conditions and genetic hereditary or acquired conditions.

The human stem cells established in lines by the process according to the invention are also useful for production of biological substances, such as for example recombinant proteins and viral vaccines. More precisely, the human stem cells set in line according to the invention are useful for replicating viruses, viral vectors derived from the latter, and for producing corresponding viral particles.

More precisely, the human stem cells set in line according to the invention are useful for production of dead, living or attenuated viral vaccines, recombinant or not. The vaccines produced in this way are intended for prophylactic and/or theracanic treatment of pathologies of viral aetiology, chronic acquired illnesses such as cancer and neurodegenerative conditions. Inexhaustive examples of viruses, viral vectors and corresponding viral particles are adenovirus, hepadnavirus, herpes virus, orthomyxovirus, papovirus, paramyxovirus, paramyxovirus, picornavirus, poxvirus, reovirus, and retrovirus. The virus is preferably orthomyxovirus, in particular human influenza virus. According to another preferred embodiment, the virus is paramyxovirus, and more particularly measles virus, and/or mumps virus, and/or rubella virus. According to another preferred embodiment, the virus is human retrovirus, and more particularly human immunodeficiency virus.

Alternatively, the human stem cells set in line according to the invention are useful for production of recombinant proteins, especially proteins of theracanic interest. In this respect, the cells obtained by the process according to the invention can be modified genetically, in a manner which is stable or transitory, by employing techniques at the disposition of the expert. According to a preferred embodiment protein of theracanic interest is an antibody, preferably monoclonal, humanised or chimerised.

Finally, the human stem cells set in line according to the invention are useful for conducting sanitary diagnostics tests.

1. A process for obtaining continuous lines of human stem cells that are non transformed, undifferentiated and capable of growing in the absence of feeder cells and proliferating indefinitely in culture, wherein said process comprises the following steps:
a) culturing human stem cells on a feeder cell layer in a complete culture medium comprising:
(i) animal serum or a substitute for animal serum;
(ii) an exogenic growth factor selected from the group consisting of FGF (Fibroblast Growth Factor), stem cell factor (SCF) and IGF1 (Insulin-like growth factor 1); and
(iii) an exogenic growth factor that is a receptor ligand which can form a hetero-dimer with glycoprotein gpl30;
b) successively passaging said human stem cells in a different culture medium, to create separation of said feeder cell layer, total or partial exogenic separation of said growth factors and total or partial separation of said serum;
c) optionally, selecting cellular colonies having compact morphologies and composed of cells having a heightened nucleo-cytoplasmic ratio and a prominent nucleole; and
d) establishing said continuous lines of human stem cells.

2. The process of claim 1, wherein the lines obtained in step d) are capable of growing in a culture medium devoid of growth factor.

3. (canceled)

4. The process of claim 1, wherein said human stem cells are selected from the group consisting of totipotent stem cells, pluripotent stem cells, multipotent stem cells, unipotent stem cells, and intermediary progenitor cells.

5. The process of claim 4, wherein said human stem cells are pluripotent stem cells.

6. The process of claim 1, wherein said receptor ligand is selected from the group consisting of leukaemia inhibitory factor (LIF), interleukine 11 (IL11), interleukine 6 (IL6), interleukine 6 receptor (IL6R), ciliary neurotrophic factor (CNTF), Foncostatine, and cardiotrophine.

7. The process of claim 1, wherein the complete culture medium of step a) comprises serum, FGF and LIF.

8. The process of claim 7, wherein the complete culture medium of step a) comprises serum, FGF, LIF, IL-6, IL6R, IL11, CNTF and IGF1.

9. The process of claim 1, wherein step a) further comprises dissociating the cellular clusters formed in culture, wherein the dissociation is carried out enzymatically and/or mechanically.

10. The process of claim 1, wherein the feeder cell layer of step a) is composed of fibroblasts selected from the group consisting of primary human fibroblasts, human fibroblasts set in line, primary mammalian fibroblasts, and mammalian fibroblasts set in line.

11. The process of claim 10, wherein said mammalian fibroblasts are mouse fibroblasts set in line.

12. The process of claim 1, wherein step b) comprises at least 30 successive passes in culture.

13. The process of claim 1, wherein the human stem cells are separated from said feeder cell layer, exogenic growth factors, and serum successively in one of the following orders:
i. feeder cell layer/serum/exogenic growth factors;
ii. feeder cell layer/exogenic growth factors/serum;
iii. serum/exogenic growth factors/feeder cell layer;
iv. serum/feeder cell layer/exogenic growth factors;
v. exogenic growth factors/serum/feeder cell layer; and
vi. exogenic growth factors/feeder cell layer/serum.

14. The process of claim 13, wherein the sequence of separations is: exogenic growth factors/feeder cell layer/serum.

15. The process of claim 1, wherein the separation from each of the exogenic growth factors is done by progressive decrease over several passes, preferably at least 3, of the concentration of each factor in the culture medium.

16. The process of claim 1, wherein the separation from exogenic growth factors is total.

17. The process of claim 1, wherein the separation from serum is done by employing a process selected from the group consisting of progressive dilution, progressive separation and direct separation.

18. An isolated human stem cell derived from a primary embryonic stem cell capable of being obtained by the process of claim 1, wherein said cell:
i. proliferates indefinitely in culture in a culture medium deprived of a cellular feeder layer, optionally serum and optionally exogenic growth factors;
ii. retains a normal diploid caryotype not altered by prolonged cellular culture;
iii. has a significant nucleo-cytoplasmic ratio;
iv. retains the capacity to differ to form at least one differentiated cellular type selected from a cellular type of mesodermic, ectodermic and endodermic origin; and
v. expresses at least telomerase and alkaline phosphatase.

19. The cell of claim 18, wherein said cell further expresses the transcription factor Oct3/4 and exhibits reactivity with at least one of the specific antibodies selected from the group consisting of antibodies directed against S SE A4, antibodies directed against TRA 1-60, and antibodies directed against TRA 1-81.

20. The isolated transgenic human stem cell of claim 18, wherein the genome of said cell was modified by:
i. insertion of an isolated pre-selected DNA sequence;
ii. substitution of a fragment of the genome cellular by an isolated pre-selected DNA sequence;
iii. deletion of an isolated pre-selected DNA sequence; or
iv. inactivation of an isolated pre-selected DNA sequence.

21. A method for using the human stem cells of claim 18 for virus replication, living or attenuated, recombinant or not, or viral vectors.

22. A method for using the human stem cells of claim 18 for the production of human or veterinary vaccines.

23. A method for using the human stem cells of claim 18 for the production of recombinants proteins or polypeptides, preferably of theracanic interest.

24. A method for using the human stem cells of claim 18 for conducting sanitary diagnostics tests.

25. The process of claim 5, wherein said pluripotent stem cells are embryonic stem cells (ES).

26. The process of claim 11, wherein the mouse fibroblasts set in line are STO cells.

27. The process of claim 26, wherein the STO cells are transformed.
